(19) United States
(12) Patent Application Publication
(10) Pub. No.: US 2023/0181049 A1
(43) Pub. Date: Jun. 15, 2023

(54) BLOOD PRESSURE METER AND METHOD FOR MEASURING BLOOD PRESSURE USING THE SAME

(71) Applicant: CHARMCARE CO., LTD., Seoul (KR)

(72) Inventor: Dong Hwa LEE, Yongin-si, Gyeonggi-do (KR)

(73) Assignee: CHARMCARE CO., LTD., Seoul (KR)

(21) Appl. No.: 17/912,859

(22) PCT Filed: Dec. 23, 2020
(86) PCT No.: PCT/KR2020/019031

§ 371 Date: Sep. 20, 2022

(30) Foreign Application Priority Data
Mar. 20, 2020 (KR) 10-2020-0034238

(51) Int. Cl. A61B 5/021

(52) U.S. Cl. CPC A61B 5/02108; A61B 2562/0247

(57) ABSTRACT

A blood pressure measurement system according to the present invention comprises: a sensor unit for detecting a human arterial pulse wave and a variable pressure arterial pulse wave; and a blood pressure calculation unit which calculates a blood pressure value using the human arterial pulse wave and the variable pressure arterial pulse wave, wherein the sensor unit senses the pulse wave at a part to which a variable pressure is applied in order to detect the variable pressure arterial pulse wave. Since a blood pressure value calculated from the arterial pulse wave detected at one part of a human body and the variable pressure arterial pulse wave detected at another part of the human body to which the variable pressure is applied can be output, blood pressure can be calculated faster than when using existing oscillometric sphygmomanometers which take at least 40 seconds to measure blood pressure, and thus an accurate blood pressure value can be calculated. Accordingly, the time required to calculate blood pressure is greatly reduced. In addition, according to the present invention, blood pressure values can be calculated through an easy and simple process by using a relative ratio value or a mapping arterial pulse wave that can be acquired from two types of waveforms composed of the arterial pulse wave and the variable pressure arterial pulse wave, and thus a complicated blood pressure calculation algorithm is not required.

### TECHNICAL FIELD

The present invention relates to a blood pressure meter and a method for measuring blood pressure, and more particularly, to a blood pressure measurement system capable of quickly calculating a blood pressure value by detecting an arterial wave for a short time, and a method for measuring a blood pressure using the same.

### BACKGROUND ART

Generally, blood pressure is a measure of the pressure that blood exerts on the walls of blood vessels, and the heart repeats contraction and relaxation approximately 60 to 80 times per minute. When the heart contracts and pushes blood, the pressure exerted on the blood vessels is called ‘systolic blood pressure’ and is called ‘highest blood pressure’ because the pressure is the highest. In addition, when the heart relaxes and receives blood, the blood vessel pressure is called ‘diastolic blood pressure’ and is called ‘lowest blood pressure’ because the pressure is the lowest.

In respect to the blood pressure of a normal person, the systolic blood pressure is 120 mmHg and the diastolic blood pressure is 80 mmHg. More than 1 in 4 Korean adults has high blood pressure, and after the age of 40, this ratio rapidly increases. Conversely, some patients are classified as hypotensive.

The high blood pressure is a problem because, if left uncontrolled, it can cause other life-threatening complications such as eye disease, kidney disease, arterial disease, brain disease, and heart disease and for patients at risk of complications or with complications, continuous blood pressure measurement and management should be performed.

With the growing interest in health and diseases related to adult diseases, such as hypertension, various types of blood pressure measuring devices are being developed. Blood pressure measurement methods include a Korotkoff sounds method, an oscillometric method, and a tonometric method.

The Korotkoff sounds method as a typical pressure measurement method is a method which in the process of applying sufficient pressure to the body part through which the arterial blood passes to block the blood flow and then decompressing the blood, the pressure at the moment when a pulse sound is first heard is measured as the systolic pressure and the pressure at the moment when the pulse sound disappears is measured as the diastolic pressure.

In addition, the oscillometric method and the tonometric method are methods applied to a digitized blood pressure measuring apparatus. The oscillometric method, like the Korotkoff sounds method, measures the systolic and diastolic blood pressures by sensing a pulse wave generated in the process of decompressing the body part through which the arterial blood passes at a constant rate after sufficiently pressurizing the body part through which the arterial blood passes to block the blood flow in the artery, or in the process of pressurizing the body part to increase the pressure at a constant rate.

Here, the pressure when the amplitude of the pulse wave is at a certain level may be measured as the systolic blood pressure or the diastolic blood pressure compared to the moment when the amplitude of the pulse wave is maximum, and the pressure when the rate of change of the pulse wave amplitude is rapidly changed may be measured as the systolic or diastolic blood pressure.

In addition, in the process of decompression at a constant rate after pressurization, the systolic blood pressure is measured before the moment when the amplitude of the pulse wave is maximum, and the diastolic blood pressure is measured later than the moment when the amplitude of the pulse wave is maximum. Conversely, in the process of pressurization at a constant rate after pressurization, the systolic blood pressure is measured after the moment when the amplitude of the pulse wave is maximum, and the diastolic blood pressure is measured earlier than the moment when the amplitude of the pulse wave is maximum.

The tonometric method is a method in which a predetermined pressure of a size that does not completely block blood flow in an artery is applied to the body part, and the blood pressure can be continuously measured using the size and shape of the generated pulse wave.

As described above, a device that measures blood pressure in various ways, that is, a blood pressure meter, is the most basic medical device for measuring blood pressure, which is the basis of a health index, and is provided almost necessarily provided in general clinics and hospitals, and widely used for individual blood pressure measurement in a home and a sports center.

Most currently used blood pressure meters are designed to measure blood pressure on the upper arm, which is similar to the height of the heart, but a product is also being developed, which can measure the blood pressure in a body part such as a wrist or a finger for carrying and measurement convenience. The above-described wrist blood pressure meter or finger blood pressure meter is smaller in size than the upper arm blood pressure meter, so it is convenient to carry and easy to measure at any time.

On the other hand, in a conventional blood pressure meter that measures blood pressure using arterial waves, for example, an oscillometric blood pressure meter detects multiple arterial pulses and measures the blood pressure, so it takes 40 seconds or more to measure the blood pressure.

### DISCLOSURE

### Technical Problem

The present invention relates to a blood pressure meter which measures blood pressure, and has been made in an effort to provide a blood pressure meter capable of quickly calculating a blood pressure value by detecting two types of pulse waves and a method for measuring blood pressure using the same.

### Technical Solution

An aspect of the present invention provides a blood pressure meter including: comprises: a sensor unit detecting a body arterial wave and a variable pressure arterial wave; and a blood pressure calculation unit calculating a blood pressure value by using the body arterial wave and the variable pressure arterial wave detected by the sensor unit, in which the sensor unit is capable of detecting a pulse wave at a site to which variable pressure is applied to detect the variable pressure arterial wave.

More specifically, one aspect of the present invention described above provides a blood pressure meter including: a sensor unit capable of detecting an arterial wave from one site of a body and detecting a variable pressure arterial wave from the other one site to which variable pressure is applied; and a blood pressure calculation unit calculating a blood pressure value by using the body arterial wave and the variable pressure arterial wave detected by the sensor unit.

The sensor unit is capable of simultaneously measuring the body arterial wave and the variable pressure arterial wave at different positions. The sensor includes a first sensor detecting the body arterial wave, and a second sensor detecting the variable pressure arterial wave at a different position from the first sensor.

The second sensor may be formed by a pressure sensor. More specifically, an air pressure sensor may be applied as the second sensor. In addition, the second sensor may detect the variable pressure arterial wave at a different position from the first sensor.

The sensor unit may include a first sensor detecting the body arterial wave at a diastolic blood pressure or less and a second sensor detecting the variable pressure arterial wave having a pulse wave of a diastolic blood pressure or less.

The blood pressure meter may further include a pulse wave processing unit calculating a relative ratio value of a change amount of the variable pressure arterial wave to a change amount of the body arterial wave measured by the sensor unit. In this case, the blood pressure calculation unit may calculate the blood pressure value by using the relative ratio value. For example, the blood pressure calculation unit may set a highest value of the relative ratio value as a highest variable pressure value, and determine the systolic blood pressure and the diastolic blood pressure based on the highest variable pressure value.

The blood pressure meter may further include a pulse wave processing unit calculating a mapped arterial wave by mapping the body arterial wave to the variable pressure arterial wave measured by the sensor unit. In this case, the blood pressure calculation unit may calculate the blood pressure value by using the mapped arterial wave. For example, the pulse wave processing unit calculates the mapped arterial wave by mapping the body arterial wave to a predetermined position based on a deformation time point of the variable pressure arterial wave.

The blood pressure meter may further include a pressurization device applying the variable pressure to a measurement site of the variable pressure arterial wave.

The pressurization device is capable of increasing or decreasing pressure for generation of the variable pressure, and the sensor unit may detect the variable pressure arterial wave during the pressure increase or decrease by the pressurization device.

The pressurization device may include at least one selected from the group consisting of a compression band, an air bag, a tightening device, a thermal expansion material, a shape change alloy, a hole, a solenoid valve, and an air pump. In other words, the pressurization device may be implemented by one or more two or more appropriate combinations of a compression band, an air bag, a tightening device, a thermal expansion material, a shape change alloy, a hole, a solenoid valve, and an air pump.

In addition, the sensor unit may include a sensor selected from the group consisting of a pressure sensor, an optical sensor, and an impedance sensor measuring impedance of a blood vessel. In other words, implementation of the sensor unit may be applied to at least one sensor such as a pressure sensor, an optical sensor, and an impedance sensor measuring impedance of a blood vessel.

The pressure sensor may include a sensor selected from the group consisting of an air pressure sensor, a film-type pressure sensor, and a strain gauge.

The sensor unit may include a first sensor and a second sensor capable of detecting the arterial waves at different sites, respectively, and any one of the first sensor and the second sensor may be applied as a sensor that measures the variable pressure arterial wave at a site under variable pressure. The sensor unit, e.g., the first sensor may measure the body arterial wave at a site under isobaric pressure.

Another aspect of the present invention provides a method for measuring blood pressure using a blood pressure meter having a sensor unit detecting an arterial wave, which includes a blood pressure calculating step of calculating, by a processor calculating blood pressure, the blood pressure by using a body arterial wave and a variable pressure arterial wave detected by the sensor unit.

The method for measuring blood pressure may further include a pulse wave detecting step of detecting the body arterial wave and the variable pressure arterial wave by using the sensor unit. In the pulse wave detecting step, the body arterial wave and the variable pressure arterial wave are detected simultaneously, i.e., at the same time.

Before the blood pressure calculating step, a pulse wave processing step of calculating a relative ratio value of a change amount of the variable pressure arterial wave to a change amount of the body arterial wave measured by the sensor unit may be conducted, and in the blood pressure calculating step, the blood pressure value may be calculated by using the relative ratio value. For example, the blood pressure calculating step may include setting a highest value of the relative ratio value as a highest variable pressure value, and determining the systolic blood pressure and the diastolic blood pressure based on the highest variable pressure value.

Before the blood pressure calculating step, a pulse wave processing step of calculating a mapped arterial wave by mapping the body arterial wave to the variable pressure arterial wave measured by the sensor unit may be conducted, and the blood pressure calculating step may include calculating the blood pressure value by using the mapped arterial wave. For example, the pulse wave processing step may include calculating the mapped arterial wave by mapping the body arterial wave based on a deformation time point of the variable pressure arterial wave.

The method for measuring blood pressure may further include a pressure varying step for controlling pressurization force for a measurement site of the variable pressure arterial wave while the sensor measures the variable pressure arterial wave.

### Advantageous Effects

According to the present invention, blood pressure value can be calculated and output from a human arterial wave detected from one part of the body and an arterial wave detected from another part of the body to which the variable pressure is applied (variable pressure arterial wave), so compared with the conventional oscillometric blood pressure meter that takes 40 seconds or more to measure the blood pressure, the blood pressure is calculated more quickly to calculate an accurate blood pressure value, thereby greatly reducing the time required for calculating the blood pressure. In addition, according to the present invention, a blood pressure value can be calculated through an easy and simple process using a relative ratio value or a mapping arterial wave obtainable from two waveforms consisting of a human arterial wave and a variable pressure arterial wave, so a complex blood pressure calculation algorithm is not required.

### DESCRIPTION OF DRAWINGS

Features and advantages of the present invention can be well appreciated with reference to drawings described below jointly with a detailed description for embodiments of the present invention to be described below, and the drawings are:

FIG. 1 is a block diagram illustrating a configuration of a blood pressure meter according to the present invention;

FIG. 2 is a diagram schematically illustrating an embodiment of the blood pressure meter according to the present invention;

FIG. 3 is a diagram illustrating a blood pressure measurement method by the blood pressure meter illustrated in FIG. 2;

FIG. 4 is a diagram schematically illustrating another embodiment of the blood pressure meter according to the present invention;

FIG. 5 is a diagram illustrating the blood pressure measurement method by the blood pressure meter illustrated in FIG. 4;

FIG. 6 is a diagram schematically illustrating yet another embodiment of the blood pressure meter according to the present invention;

FIG. 7 is a diagram illustrating a blood pressure measurement method by the blood pressure meter illustrated in FIG. 6;

FIG. 8 is a diagram schematically illustrating still yet another embodiment of the blood pressure meter according to the present invention;

FIG. 9 is a diagram schematically illustrating still yet another embodiment of the blood pressure meter according to the present invention;

FIG. 10 is a diagram schematically illustrating still yet another embodiment of the blood pressure meter according to the present invention;

FIG. 11 is a flowchart schematically illustrating an embodiment of a method for measuring blood pressure according to the present invention;

FIG. 12 is a graph for describing an embodiment of the method for measuring blood pressure according to the present invention;

FIG. 13 is a flowchart schematically illustrating another embodiment of the method for measuring blood pressure according to the present invention; and

FIG. 14 is a graph for describing another embodiment of the method for measuring blood pressure according to the present invention.

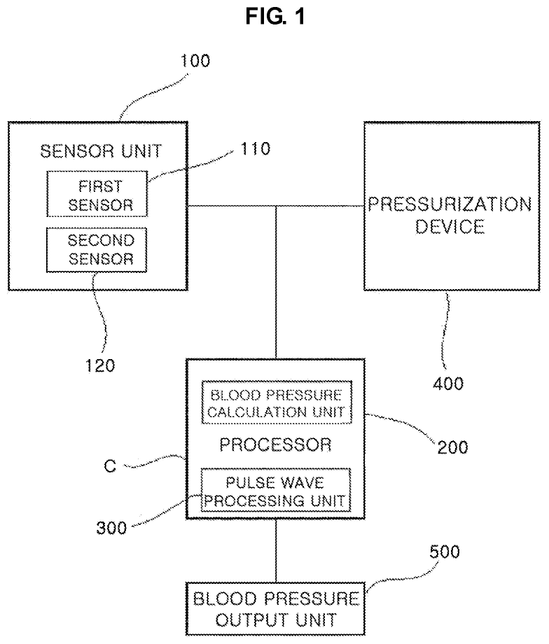

### MODES FOR THE INVENTION

Hereinafter, preferred embodiments of the present invention, in which a purpose of the present invention can be realized in detail will be described with reference to the accompanying drawings. In describing the embodiments, the same name and the same reference numeral are used with respect to the same component and the resulting additional description will be omitted.

Terms used in the present invention are used to describe the embodiments of the present invention, and are not intended to limit the present invention. For example, terms including an ordinal number, such as “first” and “second”, may be used to distinguish the elements of the same name from each other, but do not define or limit the number of elements.

In addition, it should be understood that, when it is described that a component is “connected to” or “accesses” another component, the component may be directly connected to or access the other component, but a connection relationship in which there is the other component, i.e., a relationship in which the other component is indirectly connected is also included.

In the present invention, it should be understood that term “include” or “have”indicates that a feature, a number, a step, an operation, a component, a part or the combination thereof described in the specification is present, but does not exclude a possibility of presence or addition of one or more other features, numbers, steps, operations, components, parts or combinations thereof.

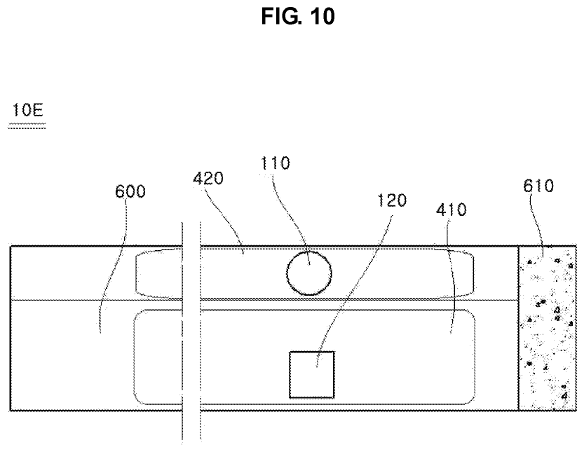

Referring to FIGS. 10 to 10, embodiments of the present invention relate to a blood pressure meter including a sensor unit 100 detecting an arterial wave of a body, such as a body arterial wave, and a variable pressure arterial wave, and a blood pressure calculation unit 200 calculating blood pressure from a signal detected by the sensor unit 100, i.e., the body arterial wave and the variable pressure arterial wave, and a method for measuring blood pressure using the same. The sensor unit 100 for an embodiment of the blood pressure meter according to the present invention has a configuration capable of detecting a pulse wave at a site to which variable pressure is applied to detect the variable pressure arterial wave, that is, a component capable of detecting the variable pressure arterial wave at a site under the variable pressure.

More specifically, the sensor unit 100 of the blood pressure meter according to an embodiment of the present invention described above is a bio signal detection unit which is capable of detecting the arterial wave from one site of the body and capable of detecting the variable pressure arterial wave from the other site to which the variable pressure is applied. In addition, the blood pressure calculation unit 200 is a component that calculates the blood pressure value by using the signal (body arterial wave and variable pressure arterial wave) detected by the sensor unit 200.

The sensor unit 100 may simultaneously measure the above-described human arterial wave and the variable pressure arterial wave at different positions. To this end, the sensor unit 100 may include a first sensor 110 for detecting the body arterial wave and a second sensor 120 for detecting the variable pressure arterial wave, and the second sensor 120 measures a bio signal, that is, the above-described variable pressure arterial wave at a position different from the first sensor 110.

In other words, the first sensor 110 and the second sensor 120 simultaneously measure the aforementioned arterial wave (body arterial wave) and variable pressure arterial wave at different positions of the body, respectively. For example, the first sensor 110 detects the arterial wave at a site under isobaric pressure, more specifically, a site to which constant pressure is applied. In addition, the second sensor 120 detects the aforementioned variable pressure arterial wave at a site different from a measurement position of the first sensor 110. In this case, the second sensor 120 detects the above-described variable pressure arterial wave at a site to which the variable pressure is applied, that is, a site to which external force is changed.

In embodiments of the present invention, the sensor unit 100 measures one arterial wave at the site under the isobaric pressure, and measures the other variable pressure arterial wave at a site under variable pressure, that is, a pressure change environment. That is, the sensor unit 100 is capable of detecting the body arterial wave at the site (e.g., a site pressurized at the isobaric pressure or to which the external force is not applied), and detecting the variable pressure arterial wave at the site under the variable pressure. In addition, the first sensor 110 and the second sensor 120 may detect the arterial wave and the variable pressure arterial wave simultaneously, i.e., at the same time. Of course, the body arterial wave and the variable pressure arterial wave may be sequentially measured at the same position.

As described above, the sensor unit 100 may include the first sensor 110 and the second sensor 120 capable of detecting the arterial waves at different sites, respectively, and any one of the first sensor and the second sensor may measure the variable pressure arterial wave at the site under the variable pressure, and in the embodiment, there is an example in which a detection sensor of the variable pressure arterial wave is applied as the second sensor 120.

The arterial wave (body arterial wave) may be measured at diastolic blood pressure or less, and the variable pressure arterial wave may include a pulse wave of the diastolic blood pressure or more. The body arterial wave may be a pulse wave measured when the external pressure applied to the artery is equal to or less than the diastolic blood pressure, for example, a pulse wave measured when the arterial wave is not deformed by the external pressure. In addition, the variable pressure arterial wave may be a pulse wave measured when the external pressure applied to the artery is equal to or less than the diastolic blood pressure, for example, a pulse wave measured when the arterial wave is deformed by the external pressure.

As the first sensor 110 and the second sensor 120, an optical sensor such as a pressure sensor and an optical blood flow meter (PPG sensor), and an impedance sensor for measuring the impedance of blood vessels may be applied. In addition, the pressure sensor may include at least one of a pneumatic sensor, a film-type pressure sensor, and a strain gauge. Since the above-described sensors themselves are known, an additional description thereof will be omitted.

In addition, the blood pressure calculation unit 200 calculates the blood pressure value using a relative ratio value or a mapped arterial wave, as described later.

More specifically, the blood pressure meter according to the embodiment includes a pulse wave processing unit 300 calculating the above-described relative ratio value or mapped arterial wave from the arterial wave (body arterial wave) and the variable pressure arterial wave.

That is, the pulse wave processing unit 300 may calculate a ratio value of a variation amount of the variable pressure arterial wave to the variation amount of the body arterial wave measured by the sensor unit 100, that is, the above-described relative ratio value.

In addition, the blood pressure calculation unit 200 may calculate the blood pressure value using the above-described relative ratio value. For example, the blood pressure calculation unit 200 may set a maximum value of the relative ratio value as a maximum variable pressure value, and determine the systolic blood pressure and the diastolic blood pressure based on the maximum variable pressure value.

As another example, the pulse wave processing unit 300 may also calculate the mapped arterial wave by mapping the variable pressure arterial wave measured by the sensor unit 100 to the body arterial wave. In this case, the blood pressure calculation unit 200 may calculate the blood pressure value by using the mapped arterial wave. More specifically, the pulse wave processing unit 300 may calculate the mapped arterial wave by mapping the body arterial wave to the variable pressure arterial wave based on a deformation time point of the variable pressure arterial wave.

The blood pressure meter 10 may further include a pressurization device 400 for pressurizing the variable pressure to a measurement site of the variable pressure arterial wave, i.e., a site (a measurement position of the second sensor) where the signal is detected by the second sensor 120.

As in a first embodiment to be described below, the variable pressure may be implemented manually while an examinee pressurizes or presses the measurement site by the second sensor 120 by himself/herself, and the variable pressure may be automatically implemented by the pressurization device 400.

The pressurization device 400 is capable of increasing the pressure (increasing the pressing force on an inspected portion) or decreasing the pressure (decreasing the pressing force on the inspected portion) in order to generate the above-described variable pressure, and the sensor unit 100, especially, the second sensor 120 detects the above-described variable pressure arterial wave during pressure increase or decrease of the inspected portion (measurement position of the second sensor) by the pressurization device 400.

The pressurization device 400 may include nay one component of components such as a compression band that presses the inspected portion, a tightener (e.g., a tightening device disclosed in Korean Patent Unexamined Publication Nos. 10-2018-0019325 and 10-2017-0042118) for tightening the inspected portion (a detection site of the variable pressure arterial wave), an air bag 410 (see drawings of embodiments to be described below), an air pump, a thermal expansion material, a shape change alloy such as a shape memory alloy, an air supply or air discharge hole, and a solenoid valve and components by a combination thereof.

The pressurization device 400 may include a passage guiding air to the air bag 410 and a valve (not illustrated) for opening/closing an air discharge port (an air discharge hole) for discharging air in the air bag.

The second sensor 120 measure the variable pressure arterial wave in the process of the pressure increase or decrease of the inspected portion by the pressurization device 400. For example, the second sensor 120 may measure the variable pressure arterial wave in the process of the pressure increase or decrease of the inspected portion at a predetermined ratio by the pressurization device 400. As a more specific example, while the air bag 410 that presses the inspected portion (the measurement position of the second sensor) is gradually expanded by an air supply action of the air pump or the air discharge is gradually conducted in the air bag 410 expanded by the air pump, the variable pressure arterial wave is measured by the second sensor 120.

As described above, when the detection of the body arterial wave by the first sensor 110 and the detection of the variable pressure arterial wave by the second sensor 120 are conducted, the pulse wave processing unit 300 acquires the relative ratio value or the mapped arterial wave by using the body arterial wave and the variable pressure arterial wave, and the blood pressure calculation unit 200 calculates the blood pressure value from the relative ratio value or the mapped arterial wave.

When the relative ratio value is used, the blood pressure calculation unit 200 determines on the maximum variable pressure value based on the maximum value of the relative value. In addition, the blood pressure calculation unit 200 determines the systolic blood pressure and the diastolic blood pressure based on the maximum variable pressure value.

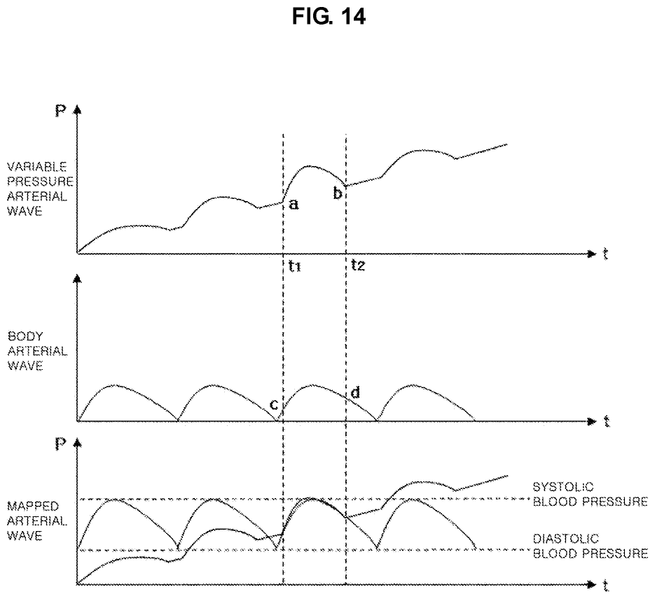

In addition, when the mapped arterial wave is used, the blood pressure calculation unit 200 calculates the mapped arterial wave by mapping the body arterial wave based on an arterial wave deformation time point (time points a and b in the graph shown at the top of FIG. 14) when the variable pressure arterial wave is measured, and calculates the blood pressure by using the mapped arterial wave. More specifically, the blood pressure calculation unit 200 determines a highest value of the mapped arterial wave as the systolic blood pressure and determines a lowest value of the mapped arterial wave as the diastolic blood pressure.

The sensor unit 100, i.e., the first sensor 110 and the second sensor 120 are controlled by a processor, i.e., a control unit C, and the pressurization device 400 is also controlled by the control unit C, and as a result, filling and exhausting of the air bag to be described below may also be performed. In addition, the blood value calculated by the method, e.g., the systolic blood pressure and the diastolic blood pressure are displayed in a blood pressure output unit 500 such as a digital monitor.

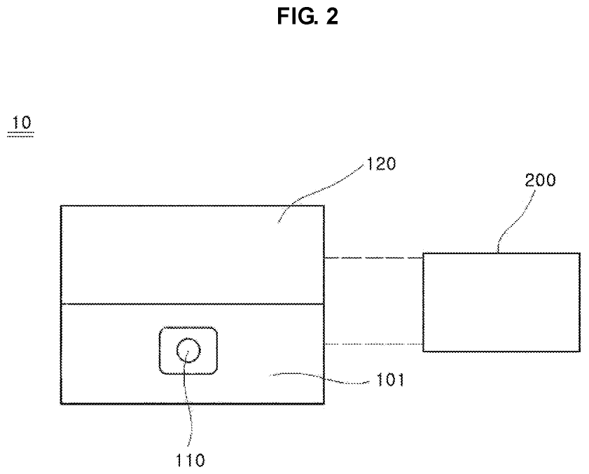

Hereinafter, specific embodiments of the blood pressure meter according to the present invention will be described with reference to FIGS. 2 to 10.

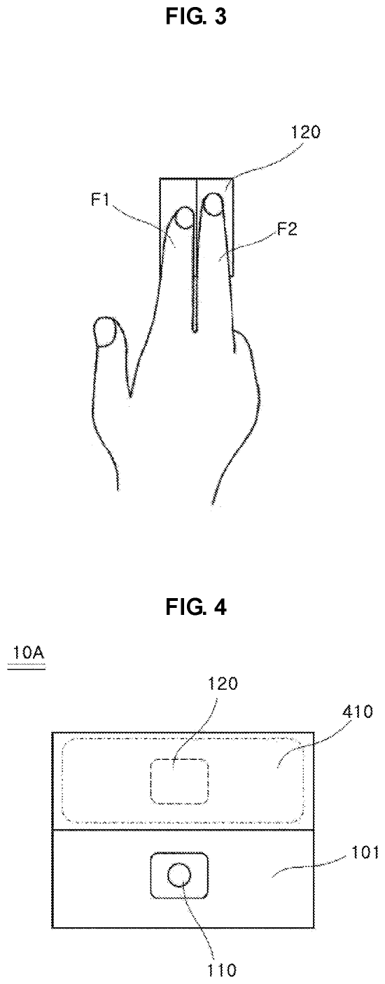

First, referring to FIGS. 2 and 3, a first embodiment 10 of the blood pressure meter according to the present invention is a blood pressure meter which detects an arterial signal, i.e., the body arterial wave and the variable pressure arterial wave in a finger, and is an example in which the first sensor 110 is formed by an optical sensor and the second sensor 120 is formed by a film-type pressure sensor. The first sensor 110 may be disposed in a finger pad 101.

The examinee places fingers F1 and F2 on a site where the first sensor 110 (optical sensor) is disposed and a site where the second sensor 120 (film-type pressure sensor) is disposed, respectively, and then places one finger F1 and brings the finger F1 into contact with constant pressure and increases the pressure while gradually pressing the finger F2 placed on the site where the second sensor 120 (film-type pressure sensor) is disposed. During this process, the first sensor 110 detects the body arterial wave and the second sensor detects the variable pressure arterial wave under the variable pressure.

The finger pad 101 may be provided as a band type which is wound on a circumference of the finger and fixable, and the second sensor 120 may also be fixed to the finger in the band type.

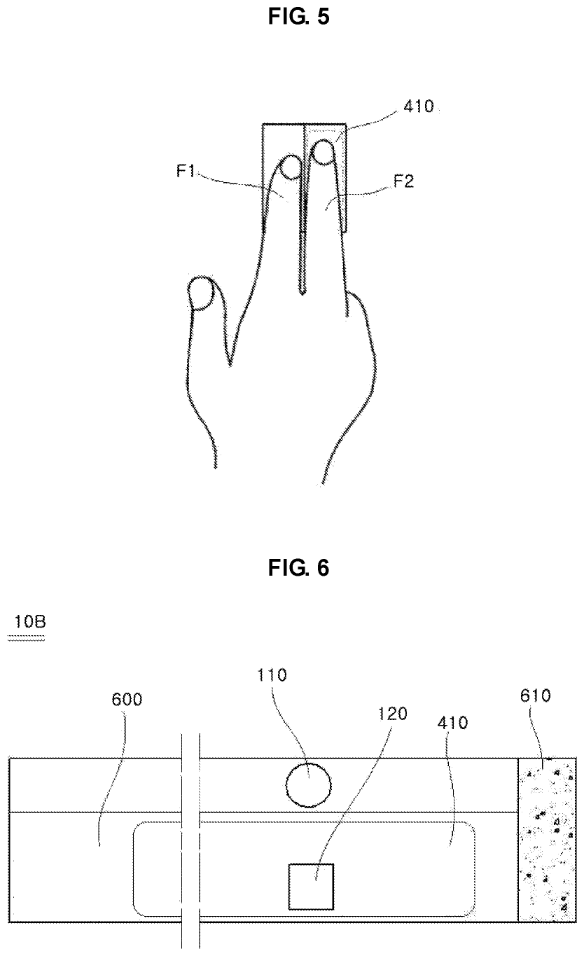

Next, referring to FIGS. 4 and 5, a second embodiment 10A of the blood pressure meter according to the present invention is also a blood pressure meter that detects an arterial signal from the finger, and is an example in which the first sensor 110 is formed by the optical sensor and the second sensor 120 is formed by the air pressure sensor, and the second sensor 120 is provided in the air bag 410. The first sensor 110 and the second sensor 120 may be wound on and fixed to the finger in the band type as in the aforementioned embodiment.

The examinee places one finger F1 on the site where the first sensor 110 (optical sensor) is disposed and makes the finger be in contact with the site and presses the air bag 410 in which the second sensor 120 (air pressure sensor) is disposed with the other finger F2. In the examine, air is discharged from an air hole (not illustrated) of the air bag 310 while the air bag 310 is pressed with the finger F2 so that predetermined pressure, e.g., pressure of 300 mmHg is formed, and in the process of the air discharge, the detection of the variable pressure arterial wave is performed by the second sensor 120 (air pressure sensor). A linear valve for controlling the flow rate may be provided in the air hole of the air bag, i.e., the air discharge hole.

In addition, when the arterial wave and the variable pressure arterial wave are performed by the first embodiment 10 and the second embodiment 10A in the above-described scheme, the pulse wave processing unit 300 acquires the relative ratio value or the mapped arterial wave and the blood pressure calculation unit 200 calculates the blood pressure by using the relative ratio value or the mapped arterial wave.

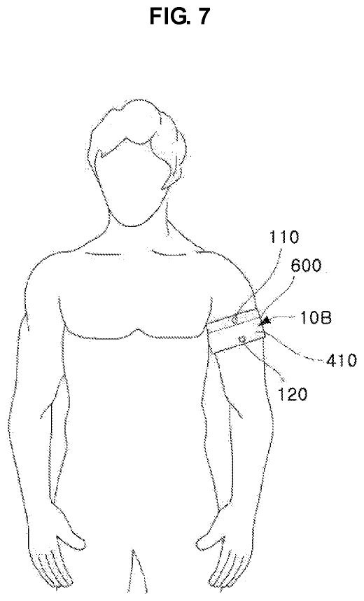

Referring to FIGS. 6 and 7, a third embodiment 10B of the blood pressure meter according to the present invention as a brachial cuff type blood pressure meter includes a first sensor 110 for detecting the body arterial wave and a second sensor 120 for the variable pressure arterial wave, and is an example in which the first sensor 110 is formed by the optical sensor and the second sensor 120 is formed by the air pressure sensor.

The first sensor 110 and the second sensor 120 are provided in a cuff belt 600 worn on an upper arm. More specifically, the air bag 410 may be provided in the cuff belt 600, and the air bag 410 may be filled by a manual or automatic pump mechanism (air pump). In addition, the second sensor 120, i.e., the air pressure sensor is provided in the air bag 410, and the first sensor 110 is provided at an external region of the air bag 410, i.e., a site not influenced by the pressure of the air bag 410.

After the brachial cuff type blood pressure meter is worn on the upper arm of the examinee by using other belt fixation means such as a Velcro 610 or a button provided in the cuff belt 600, the air is filled in the air bag 410 so that the upper arm of the examine is compressed, and pressure is not applied to a measurement site by the first sensor 110, e.g., a heart-height site from the cuff belt 600 or is in a simple contact state with the cuff belt by predetermined pressure, e.g., weak forced without fluctuation of tightening force, and a measurement site by the second sensor 120 is in a compressed state by the air bag 410.

Thereafter, the inspected portion is gradually decompressed at a predetermined ratio by the air discharge of the air bag 410, and in the process of the air discharge, the first sensor 110 detects the body arterial wave (optical arterial wave) and simultaneously, the second sensor 120 (air pressure sensor) detects the variable pressure arterial wave.

In addition, when the body arterial wave and the variable pressure arterial wave are performed by a third embodiment in the above-described scheme, the pulse wave processing unit 300 acquires the relative ratio value or the mapped arterial wave and the blood pressure calculation unit 200 calculates the blood pressure by using the relative ratio value or the mapped arterial wave.

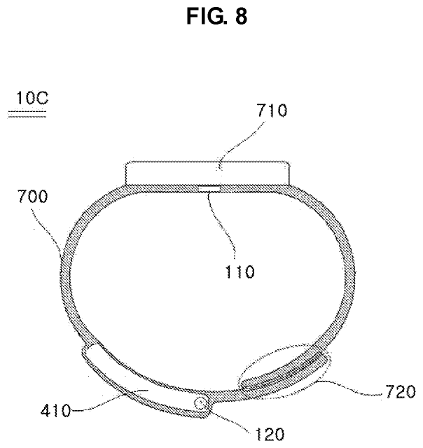

Next, referring to FIG. 8, a fourth embodiment of the blood pressure meter according to the present invention as a write blood pressure meter includes a first sensor 110 for detecting the body arterial wave and a second sensor 120 for the variable pressure arterial wave, and is an example in which the first sensor 110 is formed by the optical sensor and the second sensor 120 is formed by the air pressure sensor.

The first sensor 110 and the second sensor 120 are provided in a wrist cuff 700, i.e., a wrist strap. More specifically, the air bag 410 may be provided in the wrist cuff 700, and the air bag 410 may be filled by the manual or automatic pump mechanism (air pump). In addition, the second sensor 120, i.e., the air pressure sensor is provided in the air bag 410, and the first sensor 110 is provided at an external region of the air bag 410, i.e., a site not influenced by the pressure of the air bag 410, e.g., in a lower side of a case 710 for a display device (a blood pressure output unit) that outputs the blood pressure value. The wrist cuff 700 is connected by a strap detachable means 720 such as the Velcro or the button or a buckle.

After the wrist blood meter 10C is worn on the wrist of the examinee, the air bag 410 is filled with air at up to predetermined pressure so that the wrist of the examine is locally compressed (e.g., compression of a site through which the radial artery or the ulnar artery passes). Thereafter, the decompression is gradually made at a predetermined ratio by the air discharge of the air bag 410, and in the process of the air discharge, the first sensor 110 detects the body arterial wave (optical arterial wave) and simultaneously, the second sensor 120 (air pressure sensor) detects the variable pressure arterial wave.

In addition, when the body arterial wave and the variable pressure arterial wave are performed by a fourth embodiment in the above-described scheme, the pulse wave processing unit 300 acquires the relative ratio value or the mapped arterial wave and the blood pressure calculation unit 200 calculates the blood pressure by using the relative ratio value or the mapped arterial wave.

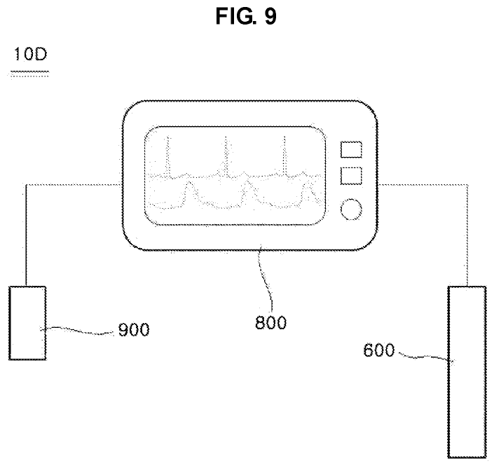

Referring to FIG. 9, a fifth embodiment 10D of the blood pressure measurement system according to the present invention as a blood pressure measurement system implemented by a patient monitoring device scheme includes an oxygen saturation measurer 900 and a brachial cuff 600 connected to a monitoring monitor 800 and separately separated from each other, and the air bag 410 and the air pressure sensor 120, i.e., the second sensor are provided in the brachial cuff 600.

The oxygen saturation measurer 900 measures the body arterial using a sensor for measuring oxygen saturation, for example, the optical sensor (first sensor; 110), and the brachial cuff 600 is a belt worn on the upper arm of the examinee, and the variable pressure arterial wave is measured in the same scheme as the third embodiment by the air bag and the air pressure sensor provided in the brachial cuff 600, i.e., the cuff belt. That is, in the embodiment, the air bag and the second sensor are provided in the brachial cuff 600, but there is no first sensor, and the oxygen saturation measurer serves as the first sensor.

In addition, when the body arterial wave and the variable pressure arterial wave are performed by a fifth embodiment in the above-described scheme, the pulse wave processing unit 300 acquires the relative ratio value or the mapped arterial wave and the blood pressure calculation unit 200 calculates the blood pressure by using the relative ratio value or the mapped arterial wave.

Next, referring to FIG. 10, a sixth embodiment 10E of the blood pressure meter according to the present invention as a brachial cuff type blood pressure meter includes a first sensor 110 for detecting the body arterial wave and a second sensor 120 for the variable pressure arterial wave, and is an example in which each of the first sensor 110 and the second pressure 120 is formed by the air pressure sensor.

The first sensor 110 and the second sensor 120 are provided a cuff belt 600 worn on an upper arm. More specifically, the first air bag 410 may be provided in the cuff belt 600, and the first air bag 410 may be filled by a manual or automatic pump mechanism (air pump). In addition, the second sensor 120, i.e., the air pressure sensor is provided in the first air bag 410, and the first sensor 110 is provided at an external region of the first air bag 410, i.e., a site not influenced by the pressure of the air bag 410.

In the embodiment, a separate air bag, i.e., a second air bag 420 is provided in the cuff belt 600, and the first sensor 110 is provided in the second air bag 420.

The brachial cuff type blood pressure meter is worn on the upper arm of the examinee by using other belt fixation means such as so called Velcro 610 or the button provided in the cuff belt 600, and then the first air bag 410 and the second air bag 420 are filed with air so that the upper arm of the examinee is compressed. Of course, the second air bag 420 may also become a structure in which air of a predetermined amount is filled and sealed.

According to the embodiment, the measurement site by the first sensor 110, e.g., the heart-height site becomes in a compressed state at predetermined pressure by the second air bag 420, and the measurement site by the second sensor 120 is in a compressed state by the air bag 410.

Thereafter, the inspected portion (the measurement site of the second sensor) is gradually decompressed at a predetermined ratio by the air discharge of the first air bag 410 and the pressure of the second air bag 420 is maintained as it is, and in the process of the air discharge, the first sensor 110 detects the body arterial wave (optical arterial wave) and simultaneously, the second sensor 120 (air pressure sensor) detects the variable pressure arterial wave.

In addition, when the body arterial wave and the variable pressure arterial wave are performed by a sixth embodiment in the above-described scheme, the pulse wave processing unit 300 acquires the relative ratio value or the mapped arterial wave and the blood pressure calculation unit 200 calculates the blood pressure by using the relative ratio value or the mapped arterial wave.

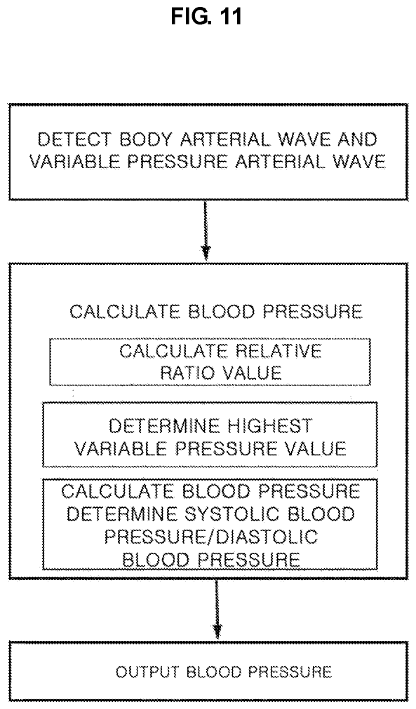

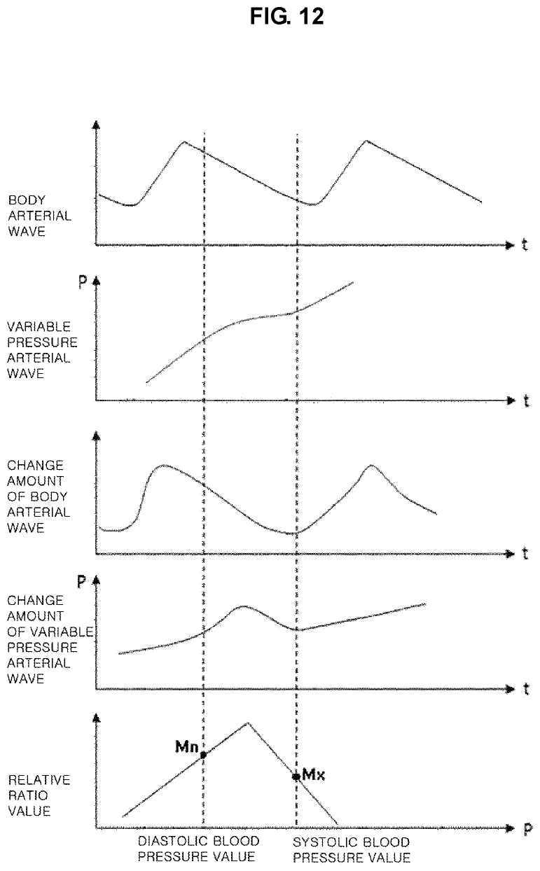

Referring to FIGS. 11 and 12, an embodiment of a blood pressure measuring method by the blood pressure meter having the sensor unit detecting the arterial wave includes a blood pressure calculating step of calculating, by the processor, i.e., the control unit C calculating the blood pressure, in particular, the blood pressure calculation unit 200, calculating the blood pressure value by using the body arterial wave and the variable pressure arterial wave detected by the sensor unit 100.

More specifically, the blood pressure calculating step of the embodiment includes a step of calculating the blood pressure value using the relative ratio value.

Of course, for calculating the blood pressure value, a pulse wave detecting step of detecting the body arterial wave and the variable pressure at different positions of the body by the sensor unit 100 is performed. For example, the body arterial wave and the variable pressure are simultaneously detected at the same time.

In the pulse wave detecting step, the variable pressure arterial may be measured in the process of the pressure increase or decrease of a site where the variable pressure arterial wave is measured. More specifically, in the pulse wave detecting step, the variable pressure arterial may be detected by sensing a pressure signal in the process of the pressure increase or decrease of a site where the variable pressure arterial wave is measured at a predetermined ratio.

A first embodiment of the blood pressure measuring method according to the present invention includes a pulse wave processing step of calculating a ratio of a change amount of the variable pressure arterial wave to the change amount of the body arterial wave detected by the sensor unit 100, i.e., the relative ratio value. In the pulse wave processing step, i.e., the calculation of the relative ratio value is conducted before the blood pressure calculating step, and in the blood pressure calculating step, a step of calculating the blood pressure value is conducted by using the relative ratio value.

More specifically, in the blood pressure calculating step, a highest variable pressure value is determined based on a highest value of the relative ratio value. In addition, the systolic blood pressure and the diastolic blood pressure are determined based on the highest variable pressure value to calculate the blood pressure value.

Referring to FIG. 12, the signal measured by the second sensor 120, e.g., the variable pressure is converted into the variable pressure arterial wave for the pressure and the first sensor 110 measures an arterial wave by an optical signal.

In the graph illustrated in FIG. 12, a top graph is a graph showing the arterial wave detected by the first sensor, i.e., the body arterial wave.

In addition, an upper second graph from the top of FIG. 12 shows the variable pressure arterial wave detected by the second sensor in a variable pressure environment, for example, the pressure decrease process, and an upper third graph shows a change amount of the arterial wave (the change amount of the body arterial wave, hereinafter, referred to as a ‘first change amount’), and an upper fourth graph is a waveform graph showing a change amount of the variable pressure arterial wave (hereinafter, referred to as a ‘second change amount’).

Last, a graph on the bottom of FIG. 12 is a graph showing a relative ratio value of the second change amount to the first change amount, i.e., a waveform graph of the relative ratio value (relative ratio wave), and a largest value (highest value) among the relative ratio values becomes a highest variable pressure value, and values at predetermined left and right points based the value becomes a systolic blood pressure value and a diastolic blood pressure value.

In other words, in an embodiment of the present invention, the relative ratio value is calculated based on the body arterial wave and the variable pressure arterial wave, and the blood pressure is calculated by using the relative ratio value. In FIG. 12, t represents the time and P represents the pressure.

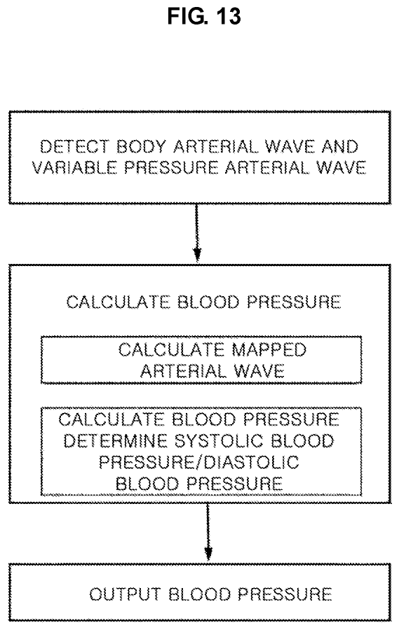

Next, referring to FIGS. 13 and 14, another embodiment (second embodiment) of the blood pressure measuring method by the blood pressure meter having the sensor unit detecting the arterial signal includes a blood pressure calculating step of calculating the blood pressure value using the body arterial wave and the variable pressure arterial wave by the processor calculating the blood pressure, i.e., the control unit C, and more specifically, the body arterial wave is mapped to the signal measured under the variable pressure, i.e., the variable pressure arterial wave to calculate the mapped arterial wave, and the blood pressure value is calculated by using the mapped arterial wave.

More specifically, the blood pressure calculating step in the embodiment includes a step of calculating the blood pressure value using the mapped arterial wave.

Of course, for calculating the blood pressure value, a pulse wave detecting step of detecting the body arterial wave and the variable pressure at different positions of the body by the sensor unit 100 is performed. For example, the body arterial wave and the variable pressure are simultaneously detected at the same time.

In the pulse wave detecting step, the variable pressure arterial may be measured in the process of the pressure increase or decrease of a site where the variable pressure arterial wave is measured. More specifically, in the pulse wave detecting step, the variable pressure arterial may be detected by sensing a pressure signal in the process of the pressure increase or decrease of a site where the variable pressure arterial wave is measured at a predetermined ratio.

The second embodiment of the blood pressure measuring method according to the present invention includes a pulse wave processing step of calculating the mapped arterial wave by mapping the body arterial wave to the variable pressure arterial wave detected by the sensor unit 100. In the pulse wave processing step, i.e., the calculation of the mapped arterial wave is conducted before the blood pressure calculating step, and in the blood pressure calculating step, a step of calculating the blood pressure value is conducted by using the mapped arterial wave.

The calculation of the mapped arterial wave is performed based on the deformation time point (deformation time point) of the variable pressure arterial wave at the time of measuring the variable pressure arterial wave. In other words, in the embodiment, the mapped arterial wave is calculated by mapping the body arterial wave measured under the isobaric pressure to a predetermine positioned based on the deformation time point of the variable pressure arterial wave at the time of measuring the variable pressure arterial wave, and the blood value is calculated by using the mapped arterial wave.

In addition, in the blood calculating step, the highest value of the mapped arterial wave is determined as the systolic blood pressure and the lowest value of the mapped arterial wave is determined as the diastolic blood pressure.

Referring to FIG. 14, the signal measured by the second sensor 120, e.g., the arterial pressure of the inspected portion is converted into the variable pressure arterial wave for the pressure and the first sensor 110 measures an arterial wave at predetermined pressure, i.e., the body arterial wave.

A top graph in the graphs illustrated in FIG. 14 is a graph in which both the pressure of the air bag itself and the pressure of the blood vessel are reflected as the pressure measured by the second sensor such as the air pressure sensor in the pressure increase process, e.g., the process of filling the air bag with air, and points a and b are time points where the variable pressure arterial wave is deformed.

In addition, the upper second graph in FIG. 14 is a graph showing the signal measured by the first sensor, i.e., the body arterial wave.

Next, a graph illustrated on the bottom of FIG. 14 as a graph showing the mapped arterial wave is a graph illustrating that the body arterial graph overlaps with the graph of the variable pressure arterial wave so that a and b which are the deformation time points of the variable pressure arterial wave of the top graph (variable arterial wave graph) overlap with the same time points (points c and d) of the graph (upper second graph) of the body arterial graph. The highest value in the mapped arterial wave is determined as the systolic blood pressure and the lowest value in the mapped arterial wave is determined as the diastolic blood pressure. For reference, at the time of mapping two arterial waves, an amplitude of the body arterial wave is adjusted so that points a and b of the variable pressure arterial wave accurately overlap with point c and d of the body arterial wave.

As described above, in the embodiments of the present invention, when the blood pressure may be calculated by using the relative ratio value and the mapped arterial wave acquired based on two bio signals, in particular, the body arterial wave and the variable pressure arterial wave, the deformation time point of the variable arterial wave is used as a reference of mapping.

Embodiments of the present invention has been described as above and a fact that the present invention can be materialized in other specific forms without departing from the gist or scope in addition to the above-described embodiments is apparent to those skilled in the art.

Therefore, the aforementioned embodiment is not limited but should be considered to be illustrative, and as a result, the present invention is not limited to the above description and may be modified within the scope of the appended claims and a range equivalent thereto.

### INDUSTRIAL APPLICABILITY

The present invention relates to a blood pressure measuring apparatus and a blood pressure measuring method for measuring blood pressure of the human body, and is an invention which is applicable in a medial device field, in particular, a blood pressure meter related technical field, and according to the preset invention, a blood pressure value can be quickly and accurately calculated based on an arterial wave.

1. A blood pressure meter comprising:
a sensor unit detecting a body arterial wave and a variable pressure arterial wave; and
a blood pressure calculation unit calculating a blood pressure value by using the body arterial wave and the variable pressure arterial wave detected by the sensor unit,
wherein the sensor unit is capable of detecting a pulse wave at a site to which variable pressure is applied to detect the variable pressure arterial wave.

2. The blood pressure meter of claim 1, wherein the sensor unit is capable of simultaneously measuring the body arterial wave and the variable pressure arterial wave at different positions.

3. The blood pressure meter of claim 2, wherein the sensor unit includes
a first sensor detecting the body arterial wave, and
a second sensor detecting the variable pressure arterial wave.

4. The blood pressure meter of claim 3, wherein the second sensor is a pressure sensor.

5. (canceled)

6. The blood pressure meter of claim 1, wherein the sensor unit
detects the body arterial wave at diastolic blood pressure or less, and
detects the variable pressure arterial wave having a pulse wave at the diastolic blood pressure or more.

7. The blood pressure meter of claim 1, further comprising:
a pulse wave processing unit calculating a relative ratio value of a change amount of the variable pressure arterial wave to a change amount of the body arterial wave measured by the sensor unit.

8. The blood pressure meter of claim 7, wherein the blood pressure calculation unit calculates the blood pressure value by using the relative ratio value.

9. The blood pressure meter of claim 8, wherein the blood pressure calculation unit sets a highest value of the relative ratio value as a highest variable pressure value, and determines the systolic blood pressure and the diastolic blood pressure based on the highest variable pressure value.

10. The blood pressure meter of claim 1, further comprising:
a pulse wave processing unit calculating a mapped arterial wave by mapping the body arterial wave to the variable pressure arterial wave measured by the sensor unit.

11. The blood pressure meter of claim 10, wherein the blood pressure calculation unit calculates the blood pressure value by using the mapped arterial wave.

12. The blood pressure meter of claim 11, wherein the pulse wave processing unit calculates the mapped arterial wave by mapping the body arterial wave based on a deformation time point of the variable pressure arterial wave.

13. The blood pressure meter of claim 1, further comprising:
a pressurization device applying the variable pressure to a measurement site of the variable pressure arterial wave.

14-18. (canceled)

19. The blood pressure meter of claim 1, wherein the sensor unit is capable of measuring the body arterial wave at a site under isobaric pressure.

20. A method for measuring blood pressure using a blood pressure meter having a sensor unit capable of detecting two arterial waves, comprising:
a blood pressure calculating step of calculating, by a processor calculating blood pressure, the blood pressure by using a body arterial wave and a variable pressure arterial wave detected by the sensor unit.

21. The method for measuring blood pressure of claim 20, further comprising
a pulse wave detecting step of detecting the body arterial wave and the variable pressure arterial wave by using the sensor unit.

22. (canceled)

23. The method for measuring blood pressure of claim 20, wherein the sensor unit
detects the body arterial wave at diastolic blood pressure or less, and
detects the variable pressure arterial wave having a pulse wave at the diastolic blood pressure or more.

24. The blood pressure meter of claim 20, wherein before the blood pressure calculating step, a pulse wave processing step of calculating a relative ratio value of a change amount of the variable pressure arterial wave to a change amount of the body arterial wave measured by the sensor unit is conducted, and
in the blood pressure calculating step, the blood pressure value is calculated by using the relative ratio value.

25. The method for measuring blood pressure of claim 24, wherein in the blood pressure calculating step, a highest value of the relative ratio value is set as a highest variable pressure value, and the systolic blood pressure and the diastolic blood pressure are determined based on the highest variable pressure value.

26. The method for measuring blood pressure of claim 20, wherein before the blood pressure calculating step, a pulse wave processing step of calculating a mapped arterial wave by mapping the body arterial wave to the variable pressure arterial wave measured by the sensor unit is conducted, and
in the blood pressure calculating step, the blood pressure value is calculated by using the mapped arterial wave.

27. The method for measuring blood pressure of claim 26, wherein in the pulse wave processing step, the mapped arterial wave is calculated by mapping the body arterial wave based on a deformation time point of the variable pressure arterial wave.

28. (canceled)
